(19) United States
(12) Patent Application Publication
(10) Pub. No.: US 2020/0037992 A1
(43) Pub. Date: Feb. 6, 2020

(54) Ultrasonic Image Processing Apparatus and Program

(71) Applicant: Hitachi, Ltd., Tokyo (JP)

(72) Inventor: Seiji OYAMA, Tokyo (JP)

(21) Appl. No.: 16/361,905

(22) Filed: Mar. 22, 2019

(30) Foreign Application Priority Data
Aug. 6, 2018 (JP) 2018-147386

(51) Int. Cl. A61B 8/00; A61B 8/06; A61B 8/08

(52) U.S. Cl. CPC A61B 8/463; A61B 8/06; A61B 8/465; A61B 8/5246; A61B 8/488

(57) ABSTRACT

A guidance image generation unit generates a guidance image in which setting position information and setting order information of each manually set representative point are marked on a schematic diagram schematically representing an organ image included in an ultrasonic image. A schema diagram schematically shows a tomographic image of the heart. A position marker is a specific example of the setting position information and a number label is a specific example of the setting order information. A plurality of position markers indicate positions corresponding to a plurality of feature points in the schema diagram schematically showing an apical three-chamber (A3C) view. A plurality of number labels indicate setting orders of the plurality of feature points.

### CROSS REFERENCE TO RELATED APPLICATION

This application claims priority to Japanese Patent Application No. 2018-147386 filed on Aug. 6, 2018, which is incorporated herein by reference in its entirety including the specification, claims, drawings, and abstract.

### TECHNICAL FIELD

The present disclosure relates to an ultrasonic image processing apparatus and a program.

### BACKGROUND

An ultrasonic diagnosis apparatus, which is one specific example of an ultrasonic image processing apparatus, is used for diagnosis of various tissues in a living body and plays an important role in diagnosis of organs such as the heart.

For example, JP 2012-100815 A discloses an ultrasonic diagnosis apparatus in which a position in an organ to which an ultrasonic tomographic image corresponds is displayed in a display image by using a schematic diagram (schema).

In addition, for example, JP 2018-51001 A discloses an ultrasonic image capturing apparatus which extracts an outline to be measured by using volume data and acquires measurement information as an anatomical structure useful for diagnosis from the outline.

Further, for example, JP 2017-196008 A discloses an ultrasonic diagnosis apparatus which determines a tracking trace point in a target range of a tracking processing with a selected trace point as a base point from a plurality of trace points constituting a trace line of a tissue in an ultrasonic image, and corrects the plurality of trace points by moving the tracking trace point so as to track a movement of the selected trace point.

### SUMMARY

In diagnosis using an ultrasonic image, an operation by a user such as a doctor or a medical technician is required in some cases. For example, a plurality of representative points are set in an ultrasonic image according to an operation by a user in some cases. For the user, it is preferable that an operation load is reduced. For example, in a case where each representative point is set manually, when the user can be informed of a setting position and a setting order of each representative point, reduction of the operation load of the user can be expected.

For reference, the ultrasonic diagnosis apparatus disclosed in JP 2012-100815 A merely displays a position in an organ to which an ultrasonic tomographic image corresponds in a display image by using a schematic diagram (schema).

An object of the present disclosure is to realize a display for guiding a user to a setting position and a setting order of each representative point manually set in an ultrasonic image.

One specific example of the present disclosure is an ultrasonic image processing apparatus including: a representative point setting unit which manually sets at least one of a plurality of representative points in an ultrasonic image based on data obtained by transmitting and receiving ultrasonic waves according to an operation by a user; and an image generation unit which generates a guidance image in which setting position information and setting order information of each manually set representative point are marked on a schematic diagram schematically representing an organ image included in the ultrasonic image.

According to one specific example of the present disclosure, a display for guiding a user to a setting position and a setting order of each representative point manually set in an ultrasonic image is realized. In addition, according to another specific example of the present disclosure, the guidance image is generated according to a type of organ image, such that it is possible to guide the user to a setting position and a setting order of each representative point appropriate for the type of organ image. Further, according to another specific example of the present disclosure, as setting position information and setting order information of each representative point manually set inside the bloodstream are marked on a schematic diagram, the user can be guided to a setting position and a setting order of each representative point inside the bloodstream, such that it is possible to reduce or eliminate confusion of the user.

### BRIEF DESCRIPTION OF DRAWINGS

Embodiment(s) of the present disclosure will be described by reference to the following figures, wherein:

FIG. 1 is a diagram showing an ultrasonic diagnosis apparatus which is one specific example of an ultrasonic image processing apparatus;

FIG. 2 is a diagram showing a specific example of a display image;

FIG. 3 is a diagram showing a specific example of a guidance image of an apical three-chamber (A3C) view;

FIG. 4 is a diagram showing a specific example of a guidance image of an apical two-chamber (A2C) view;

FIG. 5 is a diagram showing a specific example of a guidance image of an apical four-chamber (A4C) view;

FIG. 6 is a diagram showing a specific example of a guidance image of a laterally inverted apical three-chamber view;

FIG. 7 is a diagram showing a specific example of a guidance image of a laterally inverted apical two-chamber view;

FIG. 8 is a diagram showing a specific example of a guidance image of a laterally inverted apical four-chamber view;

FIG. 9 is a diagram showing a specific example of semi-auto tracing;

FIG. 10 is a diagram showing a modified example 1 of a display image;

FIG. 11 is a diagram showing a modified example 2 of a display image;

FIG. 12 is a diagram showing a modified example 3 of a display image;

FIG. 13 is a diagram showing a modified example 4 of a display image; and

FIG. 14 is a diagram showing a modified example 5 of a display image.

### DESCRIPTION OF EMBODIMENTS

First, a summary of an embodiment of the present disclosure will be described. An ultrasonic image processing apparatus according to the present disclosure includes a representative point setting unit which sets each representative point in an ultrasonic image, and an image generation unit which generates a guidance image. The representative point setting unit manually sets at least one of a plurality of representative points in an ultrasonic image based on data obtained by transmitting and receiving ultrasonic waves, according to an operation by a user. In addition, the image generation unit generates a guidance image in which setting position information and setting order information of each manually set representative point are marked on a schematic diagram schematically representing an organ image included in the ultrasonic image.

According to the embodiment, the image generation unit generates an image including a schematic diagram schematically representing the organ image included in the ultrasonic image. For example, a schematic diagram corresponding to the organ image included in the ultrasonic image may be selected among a plurality of schematic diagrams corresponding to a plurality of types of organ images to be diagnosed. For example, an ultrasonic image may be subjected to image processing to generate a schematic diagram corresponding to an organ image included in the ultrasonic image.

In addition, according to the embodiment, the image generation unit generates a guidance image in which setting position information and setting order information of each manually set representative point are marked on a schematic diagram. The setting position information is information on a position to which each representative point is set. Specific examples of the setting position information include information indicating a position (recommended position) to which each representative point should be set, information indicating a region (recommended region) in which each representative point should be set, or the like. In addition, the setting order information is information on an order in which each representative point is set. Specific examples of the setting order information include information such as a value indicating an order in which each representative point is set.

A display for guiding a user to a setting position and a setting order of each representative point manually set in an ultrasonic image is realized by the ultrasonic image processing apparatus according to the embodiment.

According to the embodiment, the image generation unit generates, for example, a guidance image in which a position marker as setting position information and a number label as setting order information are marked on a schematic diagram. For example, a position marker may indicate a setting position of each representative point, and a number label indicates a setting order of each representative point in a diagram.

In addition, according to the embodiment, the image generation unit may generate a guidance image corresponding to a type of organ image included in an ultrasonic image by marking setting position information and setting order information corresponding to the organ image on a schematic diagram selected according to the type of organ image. For example, an organ image obtained based on a plurality of different cross sections of the same organ is used in some cases. In general, even in the case of the same organ, when cross sections are different, organ images are also different. That is, even in the case of the same organ, when cross sections are different, types of organ images are also different. In a case of different organs, it may be regarded that types of organ images are different from each other. In general, when types of organ images are different, setting positions or setting orders of respective representative points in ultrasonic images including the organ images are also different. As a guidance image is generated according to a type of organ image, it is possible to guide the user to a setting position and a setting order of each representative point appropriate for the type of organ image.

In addition, according to the embodiment, the representative point setting unit may manually set one or more representative points inside the bloodstream as representative points for defining an edge of a closed region according to an operation by a user, or the image generation unit may generate a guidance image in which setting position information and setting order information of each representative point manually set inside the bloodstream are marked on a schematic diagram. In general, a structural reference does not exist inside the bloodstream, and thus the user is confused when performing manual setting of a representative point inside the bloodstream in some cases even if the user is a diagnostician such as a doctor or a medical technician. Even in this case, as setting position information and setting order information of one or more representative points inside the bloodstream are marked on a schematic diagram, the user can be guided to setting positions and setting orders of the one or more representative points inside the bloodstream, such that it is possible to reduce or eliminate confusion of the user.

According to the embodiment, the representative point setting unit may set a plurality of representative points including one or more representative points on a contour of a tissue as representative points for defining an edge of a closed region, or the image generation unit may generate a guidance image in which setting position information and setting order information of each representative point manually set on the contour of the tissue are marked on a schematic diagram.

In addition, the ultrasonic image processing apparatus according to the embodiment may set a plurality of tracking points on the edge of the closed region based on the plurality of representative points, and track movements of the plurality of tracking points over a plurality of time phases by applying a pattern matching processing between time phases based on image data of an ultrasonic image for each tracking point. Further, the ultrasonic image processing apparatus according to the embodiment may obtain vector information corresponding to one or more positions within the closed region based on movement information of each tracking point obtained by tracking movements of the plurality of tracking points set on the edge of the closed region, and Doppler information obtained from a plurality of ultrasonic beams passing through the closed region.

The summary of the ultrasonic image processing apparatus according to the embodiment has been described as above. Next, a specific example of the ultrasonic image processing apparatus according to the embodiment will be described by reference to drawings.

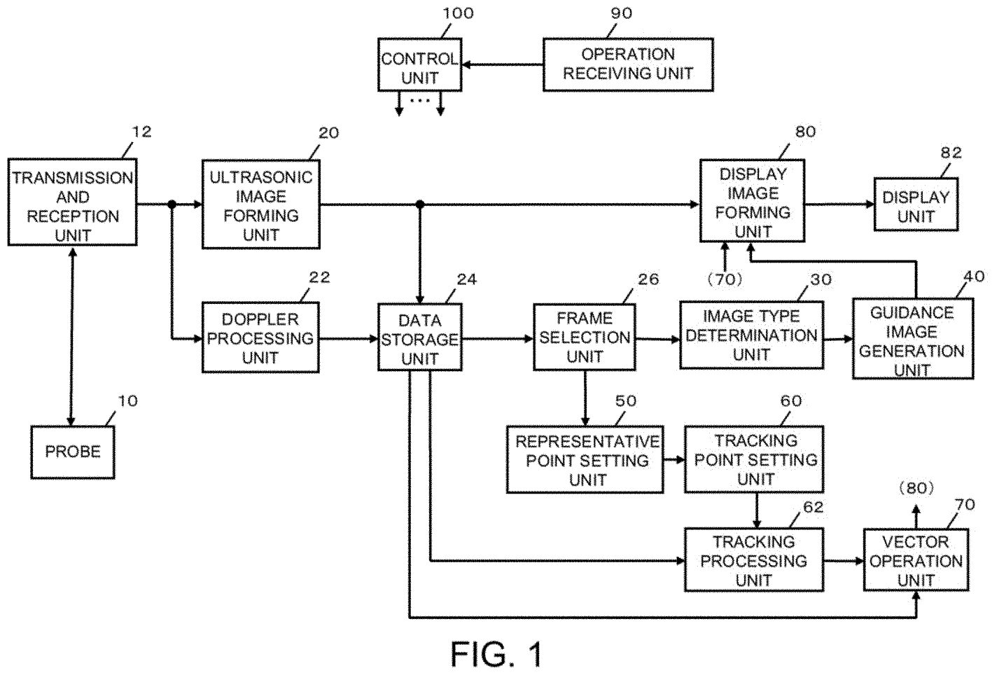

FIG. 1 is a diagram showing an ultrasonic diagnosis apparatus which is one specific example of the ultrasonic image processing apparatus according to the embodiment. The ultrasonic diagnosis apparatus shown in FIG. 1 includes components indicated by reference numerals.

A probe 10 is an ultrasonic probe transmitting and receiving ultrasonic waves in a diagnosis region including a diagnosis target. The probe 10 includes a plurality of vibration elements transmitting and receiving ultrasonic waves, and the plurality of vibration elements are subjected to a transmission control by a transmission and reception unit 12, such that a transmission beam is formed. In addition, the plurality of vibration elements receive ultrasonic waves in the diagnosis region, a signal obtained thereby is output to the transmission and reception unit 12, and the transmission and reception unit 12 forms a reception beam to obtain a reception signal (echo data). It should be noted that a technology such as a synthetic transmit aperture may be used for transmission and reception of ultrasonic waves. In addition, the probe 10 may be a three-dimensional ultrasonic probe three-dimensionally transmitting and receiving ultrasonic waves in a three-dimensional diagnosis region, or may be a two-dimensional ultrasonic probe planarly transmitting and receiving ultrasonic waves in a two-dimensional diagnosis region.

The transmission and reception unit 12 outputs a transmission signal to the plurality of vibration elements included in the probe 10, and functions as a transmission beam-former controlling the plurality of vibration elements so as to form a transmission beam. In addition, the transmission and reception unit 12 functions as a reception beam-former forming a reception beam to obtain a reception signal (echo data) based on the signal obtained from the plurality of vibration elements included in the probe 10. The transmission and reception unit 12 can be implemented by using, for example, an electric-electronic circuit (transmission and reception circuit). In this case, hardware such as an application specific integrated circuit (ASIC) or a field programmable gate array (FPGA) may be used as necessary.

An ultrasonic image forming unit 20 generates image data of an ultrasonic image based on a reception signal (echo data) obtained from the transmission and reception unit 12. The ultrasonic image forming unit 20 forms, for example, frame data of a tomographic image (B mode image) including a diagnosis target for each time phase over a plurality of time phases by performing a signal processing such as gain correction, log compression, wave detection, contour emphasis, or a filter processing with respect to the reception signal. It should be noted that a plurality of frame data spatially constituting a three-dimensional diagnosis region may be generated in a case where ultrasonic waves are three-dimensional transmitted and received and reception signals are collected from a three-dimensional diagnosis region.

A Doppler processing unit 22 measures a Doppler shift included in a reception signal obtained from an ultrasonic beam (reception beam). The Doppler processing unit 22 measures, for example, a Doppler shift generated in a reception signal of ultrasonic waves by a movement of a moving body (including the bloodstream or the like) by a known Doppler processing to obtain speed information (Doppler information) of the moving body in an ultrasonic beam direction. The Doppler processing unit 22 can be implemented by using, for example, an electric-electronic circuit (including a quadrature detection circuit or the like). In this case, hardware such as an ASIC or an FPGA may be used as necessary.

A data storage unit 24 stores an image data (frame data) of ultrasonic waves generated by the ultrasonic image forming unit 20. In addition, the data storage unit 24 stores the Doppler information (the speed information in the ultrasonic beam direction) obtained by the measurement by the Doppler processing unit 22. The data storage unit 24 can be implemented by using, for example, a storage device such as a semiconductor memory or a hard disk drive.

A frame selection unit 26 selects frame data (image data) of a time phase used for setting a representative point among frame data of a plurality of time phases stored in the data storage unit 24.

An image type determination unit 30 determines a type of an organ image (an image of a portion corresponding to an organ) included in an ultrasonic image. The image type determination unit 30 determines, for example, a type of an organ image included in the frame data of the time phase selected by the frame selection unit 26.

A guidance image generation unit 40 generates a guidance image including a schematic diagram schematically representing an organ image included in an ultrasonic image and guidance elements corresponding to a plurality of representative points. The guidance image generation unit 40 generates a guidance image in which setting position information and setting order information of each manually set representative point are marked on a schematic diagram.

The guidance image generated by the guidance image generation unit 40 is displayed on a display unit 82 through the processing by a display image forming unit 80 and is used as a display for guiding a user such as a doctor or a medical technician to a setting position and a setting order of each representative point in a case where each representative point is manually set according to an operation by the user.

A representative point setting unit 50 sets a plurality of representative points in an ultrasonic image. The representative point setting unit 50 manually sets at least one of the plurality of representative points according to an operation by the user. As a specific example of the plurality of representative points, a feature point which is a structural reference of an organ image included in an ultrasonic image may be set.

A tracking point setting unit 60 sets a plurality of tracking points on an edge of a closed region based on a plurality of representative points set by the representative point setting unit 50. The tracking point setting unit 60, for example, forms a trace line corresponding to an edge of a measurement region as a specific example of the closed region based on the plurality of representative points and sets a plurality of tracking points on the trace line.

A tracking processing unit 62 tracks movements of a plurality of tracking points over a plurality of time phases by applying a pattern matching processing between time phases based on image data of an ultrasonic image for each tracking point. The tracking processing unit 62 tracks a movement of each tracking point over a plurality of time phases (a plurality of frames) by executing a tracking processing based on image data for each tracking point.

A vector operation unit 70 derives vector information corresponding to one or more positions within a closed region based on movement information of each tracking point obtained by tracking movements of a plurality of tracking points set on an edge of the closed region, and Doppler information obtained from a plurality of ultrasonic beams passing through the closed region. The vector operation unit 70 derives, for example, a speed vector of a plurality of positions in a measurement region as a specific example of the closed region based on a tracking result of a plurality of tracking points obtained by the tracking processing unit 62 and Doppler information obtained from the data storage unit 24.

The vector operation unit 70 may derive a two-dimensional speed vector at each position in a measurement region by using speed information obtained from Doppler information in an ultrasonic beam direction and movement information obtained from a tracking result of a plurality of tracking points by, for example, a known method described in JP 2013-192643 A.

The display image forming unit 80 forms a display image displayed on the display unit 82. The display image forming unit 80 forms, for example, a display image (image data) including a guidance image generated by the guidance image generation unit 40. In addition, for example, the display image forming unit 80 may form a display image including an ultrasonic image obtained from the ultrasonic image forming unit 20 or may form a display image including vector information obtained from the vector operation unit 70.

The display unit 82 displays a display image formed by the display image forming unit 80. The display unit 82 can be implemented by using, for example, a display device such as a liquid crystal display or an organic electroluminescence (EL) display.

A control unit 100 controls the ultrasonic diagnosis apparatus in FIG. 1 overall. An indication corresponding to an operation received from the user through an operation receiving unit 90 is also reflected in the control by the control unit 100. The control unit 100 can be implemented by, for example, a combination of hardware such as a central processing unit (CPU), a processor, or a memory, and software (program) which defines an operation of the CPU, the processor, or the like. In addition, the operation receiving unit 90 can be implemented by, for example, at least one operation device among a mouse, a keyboard, a track ball, a touch panel, and other types of switches.

Among the components shown in FIG. 1, the ultrasonic image forming unit 20, the frame selection unit 26, the image type determination unit 30, the guidance image generation unit 40, the representative point setting unit 50, the tracking point setting unit 60, the tracking processing unit 62, the vector operation unit 70, and the display image forming unit 80 can each be implemented by, for example, a combination of hardware, such as a processor, and software which defines an operation of the processor or the like. In this case, hardware such as an ASIC or an FPGA may be used as necessary.

In addition, the ultrasonic diagnosis apparatus as a specific example shown in FIG. 1 can be implemented by using, for example, one or more computers. The computer includes an operation device such as a CPU, a storage device such as a memory or a hard disk, a communication device using a communication line such as Internet, a device reading data from a storage medium such as an optical disk, a semiconductor memory, or a card memory and writing data, a display device such as a display, and a hardware resource such as an operation device receiving an operation from a user.

For example, a program (software) corresponding to functions of at least some of the plurality of components denoted by reference numerals and included in the ultrasonic diagnosis apparatus shown in FIG. 1 is read by the computer and stored in a memory or the like, and the functions of at least some components of the ultrasonic diagnosis apparatus shown in FIG. 1 are implemented by a combination of the hardware resource included in the computer and the read software. For example, the program may be provided to a computer (ultrasonic diagnosis apparatus) through a communication line such as the Internet, or may be stored in a storage medium such as an optical disk, a semiconductor memory, or a card memory and provided to a computer (ultrasonic diagnosis apparatus).

An overall configuration of the ultrasonic diagnosis apparatus shown in FIG. 1 has been described as above. A diagnosis target of the ultrasonic diagnosis apparatus shown in FIG. 1 varies, and specific examples of the diagnosis target include a tissue (including the bloodstream) in a living body, a fetus in a pregnant mother, and the like. For example, the ultrasonic diagnosis apparatus in FIG. 1 may be used for diagnosis of the heart. In this regard, a specific example of a processing implemented by the ultrasonic diagnosis apparatus in FIG. 1 in a diagnosis of the heart (including the bloodstream in the heart) as one of the specific examples of the diagnosis target. It should be noted that reference numerals in FIG. 1 are used to denote the components (each unit denoted by a reference numeral) shown in FIG. 1 in the following description.

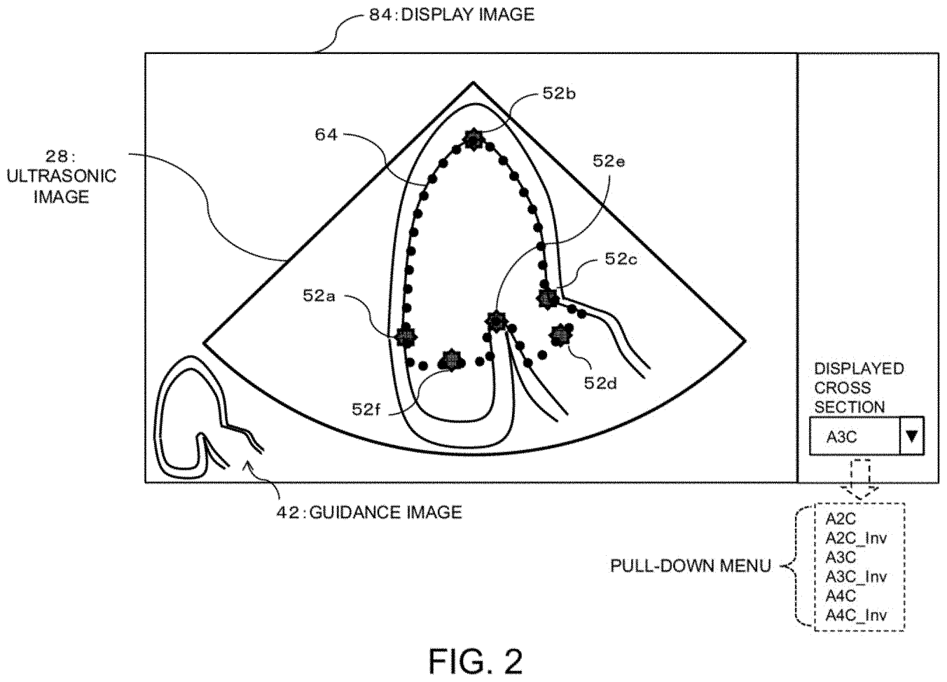

FIG. 2 is a diagram showing a specific example of a display image 84 displayed on the display unit 82. FIG. 2 shows a display image 84 including an ultrasonic image 28 and a guidance image 42.

In the specific example shown in FIG. 2, a tomographic image of the heart as a specific example of an organ image is included in the ultrasonic image 28. A plurality of feature points 52 (52a to 52f) are set in the tomographic image of the heart shown in the ultrasonic image 28.

The plurality of feature points 52 (52a to 52f) are a specific example of one or more representative points which are manually set. For example, the user such as a doctor or a medical technician indicates a setting position of each feature point 52 by operating the operation receiving unit 90 while viewing the display image 84 as the specific example shown in FIG. 2. Then, the representative point setting unit 50 sets the plurality of feature points 52 (52a to 52f) on the tomographic image of the heart according to the indication from the user obtained from the operation receiving unit 90 through the control unit 100.

It should be noted that a plurality of tracking points 64 are set in the ultrasonic image 28 shown in FIG. 2. The plurality of tracking points 64 are set by the tracking point setting unit 60. The tracking point setting unit 60 sets the plurality of tracking points 64 based on the plurality of feature points 52 (52a to 52f) as a specific example of the plurality of representative points set by the representative point setting unit 50.

Further, in the specific example shown in FIG. 2, the guidance image 42 is shown in the display image 84 together with the ultrasonic image 28. The guidance image 42 is used as a display for guiding a user to a setting position and a setting order of each feature point 52 when each feature point 52 is manually set.

Further, in the display image 84 as the specific example shown in FIG. 2, a menu display for selecting a displayed cross section is provided. The user such as a doctor or a medical technician operates, for example, the menu display for selecting a displayed cross section to select a cross section corresponding to the tomographic image of the heart in the ultrasonic image 28 from a list of displayed cross sections shown in a pull-down menu. Accordingly, a guidance image 42 corresponding to the selected cross section is displayed. For example, in the specific example shown in FIG. 2, the tomographic image of the heart in the ultrasonic image 28 is an apical three-chamber (A3C) view. The A3C is selected as the displayed cross section, and a guidance image 42 corresponding to the apical three-chamber (A3C) is shown in the display image 84.

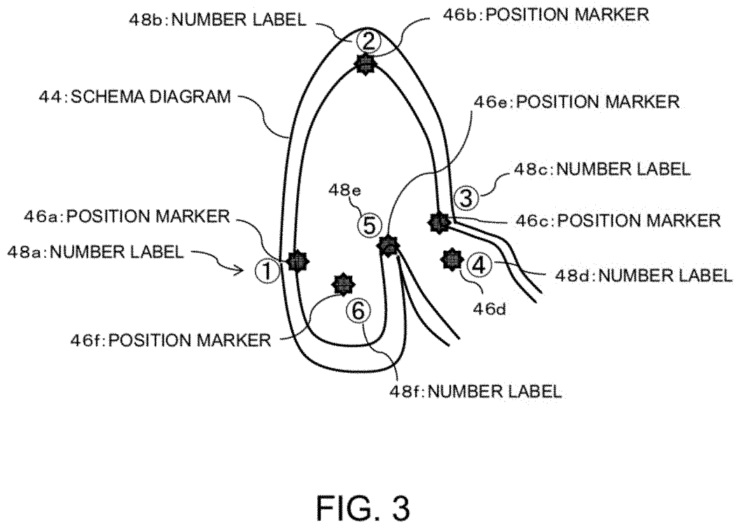

FIGS. 3 to 8 show specific examples of the guidance image 42 generated by the guidance image generation unit 40. The guidance image generation unit 40 generates a guidance image 42 in which setting position information and setting order information of each manually set representative point are marked on a schematic diagram schematically representing an organ image included in an ultrasonic image.

In each of the guidance images 42 shown in FIGS. 3 to 8, a schema diagram 44 is one of specific examples of the schematic diagram and schematically showing a tomographic image of the heart. In addition, a position marker 46 is one of specific examples of the setting position information, and a number label 48 is one of specific examples of the setting order information.

FIG. 3 shows a specific example of a guidance image 42 of an apical three-chamber (A3C) view. In the apical three-chamber (A3C) view, for example, a heart valve annulus and an apex of the heart are used as feature points, and feature points are set also in, for example, an aorta outflow passage and the left atrium. In the specific example shown in FIG. 3, a schema diagram 44 schematically showing the apical three-chamber (A3C) view is used.

In the specific example shown in FIG. 3, a plurality of position markers 46 (46a to 46f) indicate positions corresponding to a plurality of feature points in the schema diagram 44 schematically showing the apical three-chamber (A3C) view. For example, a position marker 46a indicates a position of a heart valve annulus (left), a position marker 46b indicates a position of an apex of heart, a position marker 46c indicates a position of the heart valve annulus (right), and a position marker 46e indicates a position of the heart valve annulus (middle). In addition, a position marker 46d indicates a position in an aorta outflow passage, and a position marker 46f indicates a position of the left atrium.

Further, in the specific example shown in FIG. 3, a plurality of number labels 48 (48a to 48f) indicate setting orders of the plurality of feature points. For example, a number label 48a indicates that a feature point corresponding to the heart valve annulus (left) is first in a setting order, a number label 48b indicates that a feature point corresponding to the apex of heart is second in the setting order, and a number label 48c indicates that a feature point corresponding to the heart valve annulus (right) is third in the setting order. Further, a number label 48d indicates that a feature point corresponding to the aorta outflow passage is fourth in the setting order, a number label 48e indicates that a feature point corresponding to the heart valve annulus (middle) is fifth in the setting order, and a number label 48f indicates that a feature point corresponding to the left atrium is sixth in the setting order.

For example, when an organ image included in an ultrasonic image is the apical three-chamber (A3C) view, and a plurality of feature points are manually set in the ultrasonic image, the guidance image 42 as the specific example shown in FIG. 3 is used as a display for guiding the user such as a doctor or a medical technician to a setting position and a setting order of each feature point. For example, as in the specific example shown in FIG. 2, the display image 84, in which the guidance image 42 is shown together with the ultrasonic image 28, is formed and displayed on the display unit 82.

As a result, the user can intuitively and naturally grasp a position to which each feature point is to be set in the ultrasonic image 28 and naturally understand an order in which a plurality of feature points are set, based on, for example, a correspondence between the organ image of the apical three-chamber (A3C) view included in the ultrasonic image 28 and the schema diagram 44 of the apical three-chamber (A3C) view included in the guidance image 42.

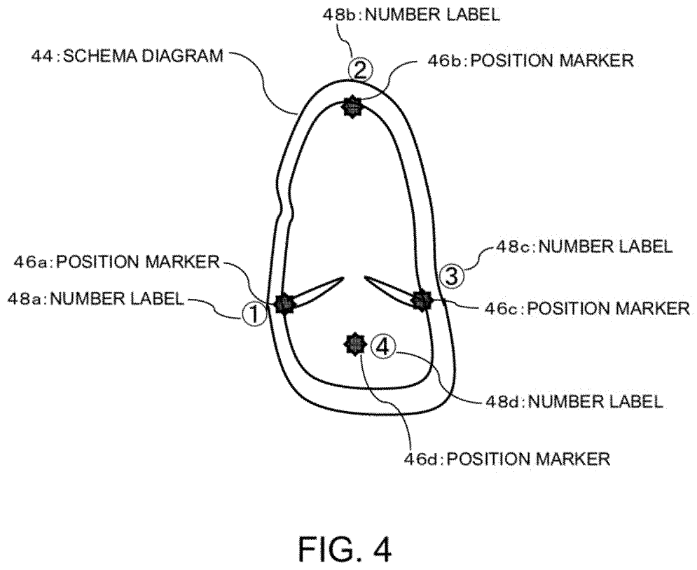

When a type of organ image included in the ultrasonic image is the apical three-chamber (A3C) view, the guidance image 42 as the specific example shown in FIG. 3 is used. FIGS. 4 to 8 show specific examples of a guidance image 42 used when the type of organ image is other than the apical three-chamber (A3C) view.

FIG. 4 shows a specific example of a guidance image 42 of an apical two-chamber (A2C) view. In the apical two-chamber (A2C) view, for example, a heart valve annulus and an apex of heart are used as feature points, and a feature point is set also in, for example, the left atrium.

In the specific example shown in FIG. 4, a schema diagram 44 schematically showing the apical two-chamber (A2C) view is used. In addition, a plurality of position markers 46 (46a to 46d) indicate positions corresponding to a plurality of feature points in the schema diagram 44 schematically showing the apical two-chamber (A2C) view. For example, a position marker 46a indicates a position of a heart valve annulus (left), a position marker 46b indicates a position of an apex of the heart, a position marker 46c indicates a position of the heart valve annulus (right), and a position marker 46d indicates a position of the left atrium. Further, a plurality of number labels 48 (48a to 48d) indicate setting orders of the plurality of feature points. For example, a number label 48a indicates that a feature point corresponding to the heart valve annulus (left) is first in a setting order, a number label 48b indicates that a feature point corresponding to the apex of heart is second in the setting order, a number label 48c indicates that a feature point corresponding to the heart valve annulus (right) is third in the setting order, and a number label 48d indicates that a feature point corresponding to the left atrium is fourth in the setting order.

For example, when an organ image included in an ultrasonic image is the apical two-chamber (A2C) view, and a plurality of feature points are manually set in the ultrasonic image, the guidance image 42 as the specific example shown in FIG. 4 is used as a display for guiding the user such as a doctor or a medical technician to a setting position and a setting order of each feature point. For example, a display image, in which the guidance image 42 in FIG. 4 is shown together with the ultrasonic image including the apical two-chamber (A2C) view, is formed and displayed on the display unit 82.

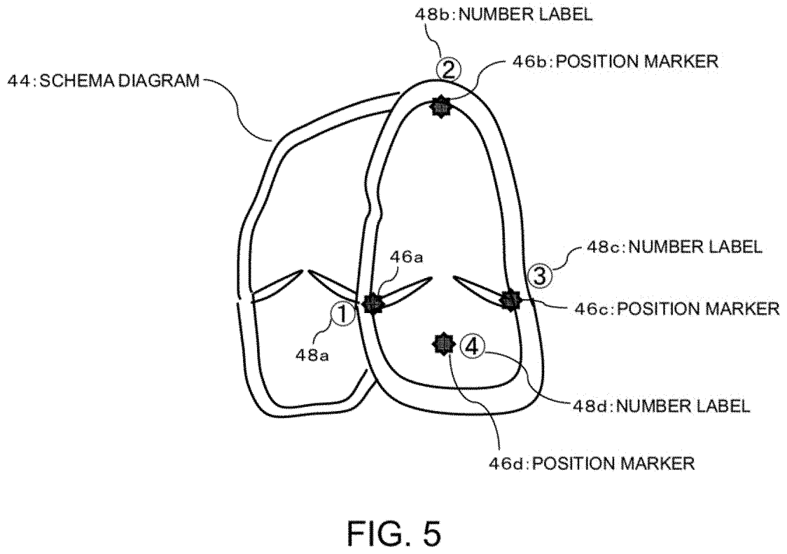

FIG. 5 shows a specific example of a guidance image 42 of an apical four-chamber (A4C) view. In the apical four-chamber (A4C) view, for example, a heart valve annulus and an apex of heart are used as feature points, and a feature point is set also in, for example, the left atrium.

In the specific example shown in FIG. 5, a schema diagram 44 schematically showing the apical four-chamber (A4C) view is used. In addition, a plurality of position markers 46 (46a to 46d) indicate positions corresponding to a plurality of feature points in the schema diagram 44 schematically showing the apical four-chamber (A4C) view. For example, a position marker 46a indicates a position of a heart valve annulus (left), a position marker 46b indicates a position of an apex of the heart, a position marker 46c indicates a position of the heart valve annulus (right), and a position marker 46d indicates a position of the left atrium. Further, a plurality of number labels 48 (48a to 48d) indicate setting orders of the plurality of feature points. For example, a number label 48a indicates that a feature point corresponding to the heart valve annulus (left) is first in a setting order, a number label 48b indicates that a feature point corresponding to the apex of heart is second in the setting order, a number label 48c indicates that a feature point corresponding to the heart valve annulus (right) is third in the setting order, and a number label 48d indicates that a feature point corresponding to the left atrium is fourth in the setting order.

For example, when an organ image included in an ultrasonic image is the apical four-chamber (A4C) view, and a plurality of feature points are manually set in the ultrasonic image, the guidance image 42 as the specific example shown in FIG. 5 is used as a display for guiding the user such as a doctor or a medical technician to a setting position and a setting order of each feature point. For example, a display image, in which the guidance image 42 in FIG. 5 is shown together with the ultrasonic image including the apical four-chamber (A4C) view, is formed and displayed on the display unit 82.

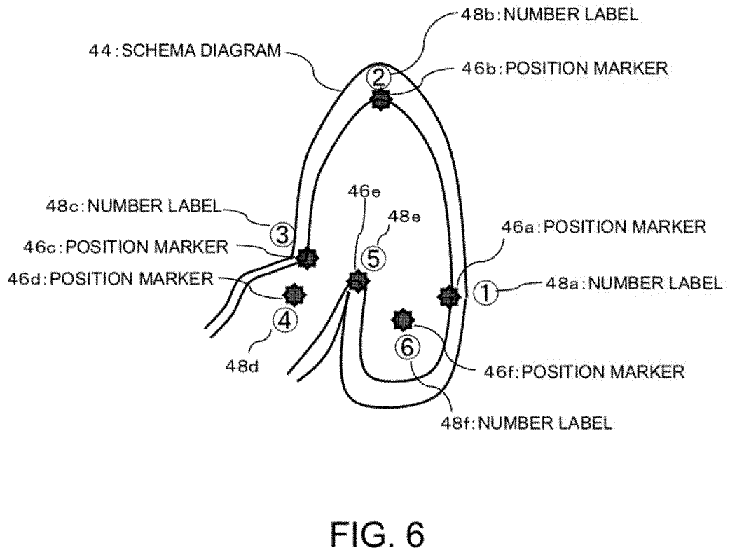

FIG. 6 shows a specific example of a guidance image 42 of a laterally inverted apical three-chamber (A3C_Inv) view. For example, when an organ image included in an ultrasonic image is the laterally inverted apical three-chamber (A3C_Inv) view, and a plurality of feature points are manually set in the ultrasonic image, the guidance image 42 as the specific example shown in FIG. 6 is used as a display for guiding the user such as a doctor or a medical technician to a setting position and a setting order of each feature point.

In the specific example shown in FIG. 6, a schema diagram 44 schematically showing the laterally inverted apical three-chamber (A3C_Inv) view is used. In addition, a plurality of position markers 46 (46a to 46f) indicate positions corresponding to a plurality of feature points in the schema diagram 44 schematically showing the laterally inverted apical three-chamber (A3C_Inv) view. For example, a position marker 46a indicates a position of a heart valve annulus (right), a position marker 46b indicates a position of an apex of heart, a position marker 46c indicates a position of the heart valve annulus (left), a position marker 46d indicates a position of an aorta outflow passage, a position marker 46e indicates a position of the heart valve annulus (middle), and a position marker 46f indicates a position of the left atrium.

Further, in the specific example shown in FIG. 6, a plurality of number labels 48 (48a to 48f) indicate setting orders of the plurality of feature points. For example, a number label 48a indicates that a feature point corresponding to the heart valve annulus (right) is first in a setting order, a number label 48b indicates that a feature point corresponding to the apex of heart is second in the setting order, a number label 48c indicates that a feature point corresponding to the heart valve annulus (left) is third in the setting order, a number label 48d indicates that a feature point corresponding to the aorta outflow passage is fourth in the setting order, a number label 48e indicates that a feature point corresponding to the heart valve annulus (middle) is fifth in the setting order, and a number label 48f indicates that a feature point corresponding to the left atrium is sixth in the setting order.

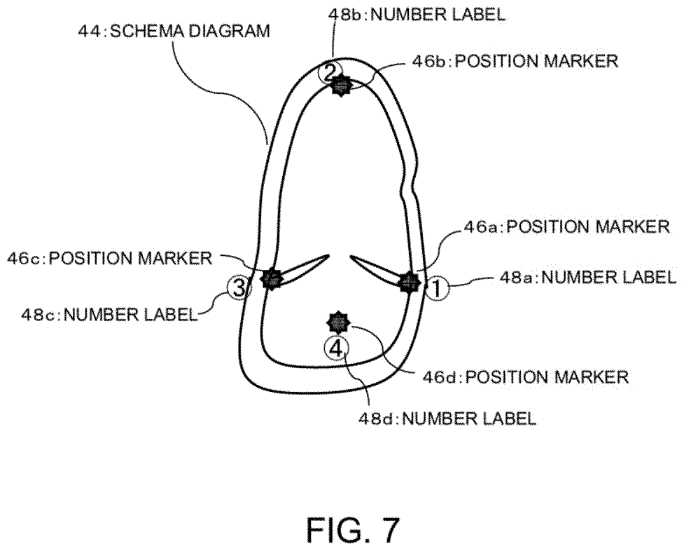

FIG. 7 shows a specific example of a guidance image 42 of a laterally inverted apical two-chamber (A2C_Inv) view. For example, when an organ image included in an ultrasonic image is the laterally inverted apical two-chamber (A2C_Inv) view, and a plurality of feature points are manually set in the ultrasonic image, the guidance image 42 as the specific example shown in FIG. 7 is used as a display for guiding the user such as a doctor or a medical technician to a setting position and a setting order of each feature point.

In the specific example shown in FIG. 7, a schema diagram 44 schematically showing the laterally inverted apical two-chamber (A2C_Inv) view is used. In addition, a plurality of position markers 46 (46a to 46d) indicate positions corresponding to a plurality of feature points in the schema diagram 44 schematically showing the laterally inverted apical two-chamber (A2C_Inv) view. For example, a position marker 46a indicates a position of a heart valve annulus (right), a position marker 46b indicates a position of an apex of the heart, a position marker 46c indicates a position of the heart valve annulus (left), and a position marker 46d indicates a position of the left atrium. Further, a plurality of number labels 48 (48a to 48d) indicate setting orders of the plurality of feature points. For example, a number label 48a indicates that a feature point corresponding to the heart valve annulus (right) is first in a setting order, a number label 48b indicates that a feature point corresponding to the apex of heart is second in the setting order, a number label 48c indicates that a feature point corresponding to the heart valve annulus (left) is third in the setting order, and a number label 48d indicates that a feature point corresponding to the left atrium is fourth in the setting order.

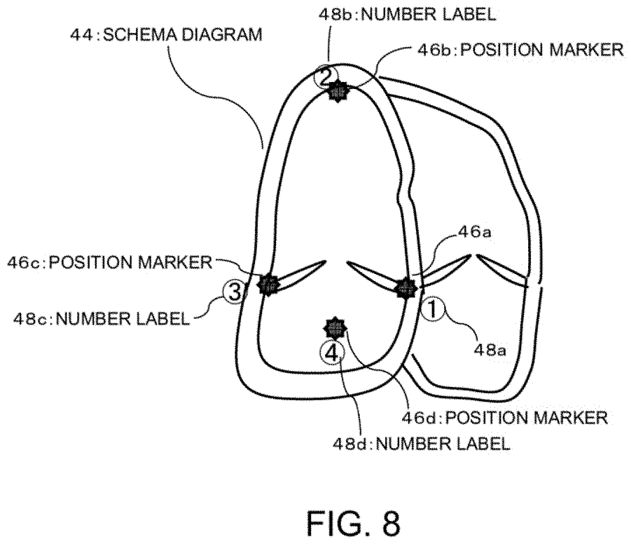

FIG. 8 shows a specific example of a guidance image 42 of a laterally inverted apical four-chamber (A4C_Inv) view. For example, when an organ image included in an ultrasonic image is the laterally inverted apical four-chamber (A4C_Inv) view, and a plurality of feature points are manually set in the ultrasonic image, the guidance image 42 as the specific example shown in FIG. 8 is used as a display for guiding the user such as a doctor or a medical technician to a setting position and a setting order of each feature point.

In the specific example shown in FIG. 8, a schema diagram 44 schematically showing the laterally inverted apical four-chamber (A4C_Inv) view is used. In addition, a plurality of position markers 46 (46a to 46d) indicate positions corresponding to a plurality of feature points in the schema diagram 44 schematically showing the laterally inverted apical four-chamber (A4C_Inv) view. For example, a position marker 46a indicates a position of a heart valve annulus (right), a position marker 46b indicates a position of an apex of the heart, a position marker 46c indicates a position of the heart valve annulus (left), and a position marker 46d indicates a position of the left atrium. Further, a plurality of number labels 48 (48a to 48d) indicate setting orders of the plurality of feature points. For example, a number label 48a indicates that a feature point corresponding to the heart valve annulus (right) is first in a setting order, a number label 48b indicates that a feature point corresponding to the apex of heart is second in the setting order, a number label 48c indicates that a feature point corresponding to the heart valve annulus (left) is third in the setting order, and a number label 48d indicates that a feature point corresponding to the left atrium is fourth in the setting order.

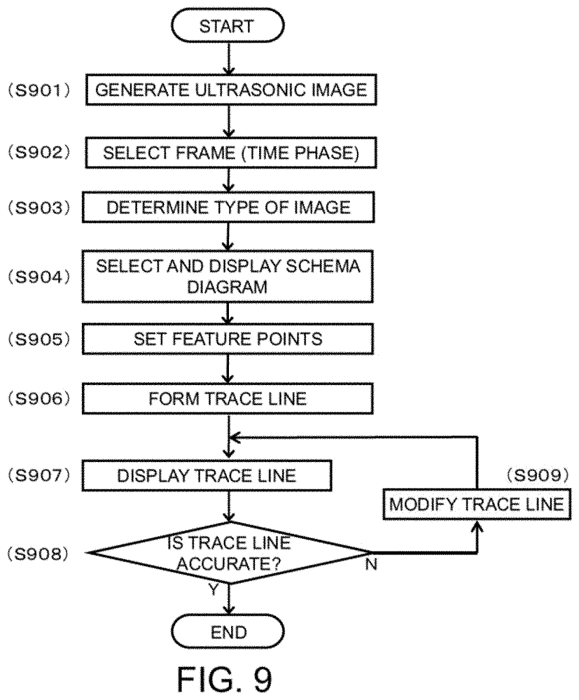

FIG. 9 is a diagram (flowchart) showing a specific example of a processing executed by the ultrasonic diagnosis apparatus in FIG. 1. FIG. 9 shows a specific example of a semi-auto tracing (a semi-automated trace line forming processing) executed by the ultrasonic diagnosis apparatus in FIG. 1. For example, when a diagnosis mode which requires the semi-auto tracing is selected, a processing in the flowchart shown in FIG. 9 is started.

Once the flowchart shown in FIG. 9 is started, first, an ultrasonic image is generated (S901). In a diagnosis of the heart, an inspector (the user such as a doctor or a medical technician) brings a wave transmitting and receiving surface of the probe 10 into contact with the skin of a subject, and adjusts a position or an orientation of the probe 10 so that an ultrasonic image (tomographic image) of the heart of the subject is displayed on the display unit 82. Then, image data (frame data) of a plurality of time phases of the heart are collected in a state where a desired tomographic image can be obtained. The collected image data of the plurality of time phases are stored in the data storage unit 24.

Next, a frame (time phase) is selected (S902). For example, image data (frame data) of a time phase used for a trace line forming processing is selected among the image data of the plurality of time phases stored in the data storage unit 24. For example, a display image showing contents of the image data of the plurality of time phases stored in the data storage unit 24 is displayed on the display unit 82, and the inspector designates image data of a desired time phase by operating the operation receiving unit 90 while viewing the display image. Then, the frame selection unit 26 selects the image data (frame data) of the time phase designated by the inspector. It should be noted that the frame selection unit 26 may also perform automatic selection (selection which does not require an instruction from the inspector) of frame data of a time phase corresponding to a particular time phase such as end-diastole.

Next, a type of image is determined (S903). The image type determination unit 30 determines, for example, a type of an organ image included in the image data (frame data) of the time phase selected by the frame selection unit 26. For example, in the case of the diagnosis of the heart, the image type determination unit 30 selects a type designated by the inspector among representative types of organ images such as an apical three-chamber (A3C) view, an apical two-chamber (A2C) view, and an apical four-chamber (A4C) view.

For example, the image type determination unit 30 may also perform automatic determination (determination which does not require an instruction from the inspector) of a type of an organ image through an image recognition processing for the image data of the time phase selected by the frame selection unit 26. The image type determination unit 30 may also perform automatic determination of a type of an organ by using, for example, a technology related to the image recognition processing described in JP 5242163 B2.

A summary of the automatic determination using the technology of JP 5242163 B2 is as follows. For example, a template (standard template) as a standard is prepared in advance for each type of organ image. For example, a standard template is prepared for each of representative types of organ images such as an apical three-chamber (A3C) view, an apical two-chamber (A2C) view, and an apical four-chamber (A4C) view. The image type determination unit 30 applies the processing specifically described in JP 5242163 B2 to target image data (image data of the time phase selected by the frame selection unit 26) to thereby template the target image data. Then, the image type determination unit 30 may perform comparison between the templated target image data and standard templates prepared in advance, in which the processing specifically described in JP 5242163 B2 is applied, and determine a standard template to which the target image data correspond (a standard template from which a difference is less than a threshold value as a result of the comparison), thereby determining a type of organ image included in the target image data.

When the type of image is determined, a schema diagram is selected and displayed (S904). For example, in the case of the diagnosis of the heart, a plurality of schema diagrams schematically representing organ images are prepared in advance for each of representative types of organ images such as an apical three-chamber (A3C) view, an apical two-chamber (A2C) view, and an apical four-chamber (A4C) view. The image type determination unit 30 selects, for example, a schema diagram corresponding to the type of organ image determined in S903 among the plurality of schema diagrams prepared in advance. Then, the schema diagram selected by the image type determination unit 30 is displayed on the display unit 82.

Next, feature points are set (S905). The representative point setting unit 50 manually sets, for example, at least one of a plurality of feature points as specific examples of a plurality of representative points according to an operation by the inspector (the user such as a doctor or a medical technician). In the manual setting, a guidance image 42 (for example, see FIGS. 3 to 8) corresponding to the schema diagram selected in S904 is used. For example, a display image 84 (for example, see FIG. 2) including the guidance image 42 corresponding to the schema diagram selected in S904 and the ultrasonic image 28 corresponding to the image data of the time phase selected in S902 is displayed on the display unit 82, and the inspector sequentially designates setting positions of the plurality of feature points in the ultrasonic image 28 according to a setting position and a setting order of each feature point about which the guidance image 42 guides while viewing the display image 84.

It should be noted that the representative point setting unit 50 may detect a setting position of at least one of the plurality of feature points in the ultrasonic image 28. The representative point setting unit 50 may interpret, for example, an image (organ image) in the image data of the time phase selected in S902 to detect a position corresponding to one or more feature points in the ultrasonic image 28 corresponding to the image data. For example, in a case of a tomographic image of the heart, the representative point setting unit 50 may detect a position of an image of a heart valve annulus portion with a relatively higher brightness in the image as a position of a feature point corresponding to the heart valve annulus.

When the feature points are set, a trace line is formed (S906). The tracking point setting unit 60 forms a trace line based on the plurality of feature points set in S905.

For example, in the case where the tomographic image of the heart in the ultrasonic image 28 is an apical three-chamber (A3C) view as shown in FIG. 2, the tracking point setting unit 60 extracts contours of the left ventricle, the left atrium, and the aorta based on the feature points 52a, 52c, and 52e corresponding to three heart valve annulus portions and the feature point 52b corresponding to an apex of heart. It should be noted that, for example, a known method such as dynamic contour modeling described in a pamphlet of WO 2011/083789 A may be used for the extraction of the contours by the tracking point setting unit 60. In addition, the tracking point setting unit 60 sets a boundary for dividing the left atrium from a contour of one side of the left atrium to a contour of the other side of the left atrium through the feature point 52f, and sets a boundary for dividing the aorta from a contour of one side of the aorta to a contour of the other side of the aorta through the feature point 52d. Accordingly, in the specific example shown in FIG. 2, for example, a trace line constituted by the contour of the left ventricle, the contour of the left atrium, the contour of the aorta, the boundary for dividing the left atrium, and the boundary for dividing the aorta is formed.

It should be noted that a trace line corresponding to each of types of organ images is formed when the type of organ image in the ultrasonic image 28 is an apical two-chamber (A2C) view, and an apical four-chamber (A4C) view, or the like.

When the trace line is formed, the formed trace line is displayed on the display unit 82 (S907), and the inspector (the user such as a doctor or a medical technician) checks whether or not the trace line is accurate (S908). When the trace line is not accurate, the inspector modifies a position or a shape of the trace line displayed on the display unit 82 by, for example, operating the operation receiving unit 90 (S909). When the trace line is accurate, the processing (semi-auto tracing) shown in FIG. 9 ends.

For example, when the trace line is formed by the processing shown in FIG. 9, the tracking point setting unit 60 sets a plurality of tracking points on the trace line. The tracking point setting unit 60 sets, for example, approximately 100 tracking points on the trace line. Accordingly, a plurality of tracking points 64 are set along the trace line in the ultrasonic image 28 as in the specific example shown in FIG. 2.

When the plurality of tracking points are set, the tracking processing unit 62 executes tracking processing based on, for example, image data for each tracking point. The tracking processing unit 62 tracks a movement of each tracking point over a plurality of time phases based on, for example, the image data of the plurality of time phases stored in the data storage unit 24 as a processing target. The tracking processing unit 62 tracks movements of a plurality of tracking points over a plurality of time phases by, for example, applying pattern matching processing between time phases based on image data for each tracking point. Accordingly, in the case of diagnosis of the heart, for example, movement information of the heart wall can be obtained based on the plurality of tracking points.

The vector operation unit 70 may derive a two-dimensional speed vector at each position in a measurement region by using speed information obtained from Doppler information in an ultrasonic beam direction and the movement information obtained from a tracking result of the plurality of tracking points by, for example, the known method described in JP 2013-192643 A. For example, the vector operation unit 70 may execute a processing (vector flow mapping (VFM)) of forming distribution of two-dimensional speed vectors by deriving a speed vector for each of a plurality of sample points in a coordinate system for operation corresponding to a space in which ultrasonic waves are transmitted and received.

For example, the display image forming unit 80 forms a display image including the distribution of the speed vectors formed by the vector operation unit 70 and the display image is displayed on the display unit 82. Accordingly, in the case of diagnosis of the heart, for example, it is possible for the inspector to visually check a state of the bloodstream in the heart.

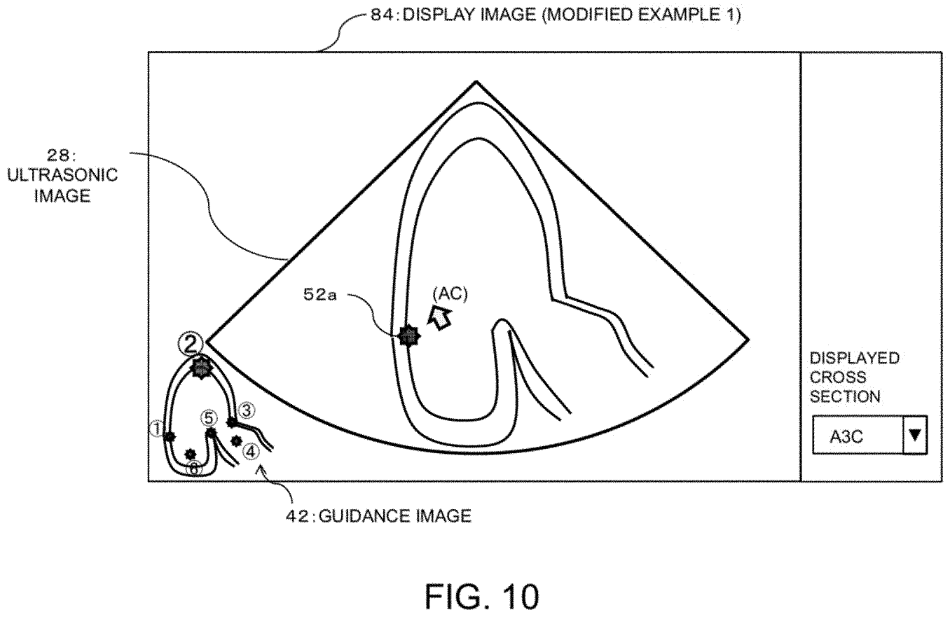

FIGS. 10 to 14 are diagrams showing specific examples of a display image 84 displayed on the display unit 82. FIGS. 10 to 14 show specific example of a display image 84 including an ultrasonic image 28 and a guidance image 42.

In a modified example 1 shown in FIG. 10, a position and an order of a feature point which should be set next by the user such as a doctor or a medical technician in manual setting are emphatically marked. For example, a position marker and a number label corresponding to a feature point which should be set next are emphatically marked on the guidance image 42. For example, as in the specific example shown in FIG. 10, when a position of the first feature point 52a is set, a position marker and a number label corresponding to the second feature point which should be set next are enlarged to be emphasized in the guidance image 42. For example, a position marker and a number label may also be emphatically marked in a manner in which a color, a brightness, or the like is changed.

Then, the user such as a doctor or a medical technician moves an arrow-shaped cursor AC shown in the display image 84 to a desired position by, for example, operating the operation receiving unit 90 to designate a position of a feature point which should be set next.

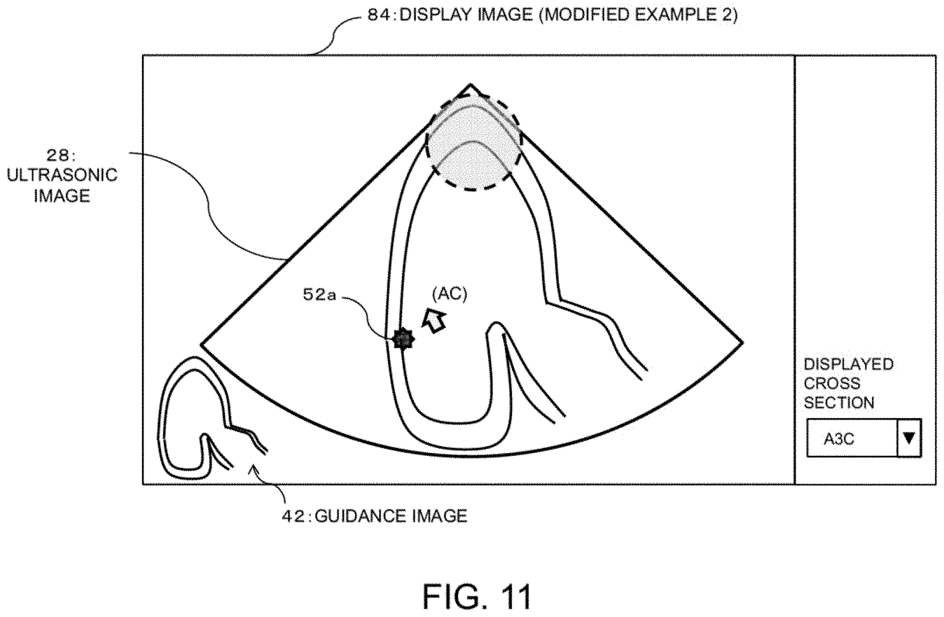

In a modified example 2 shown in FIG. 11, the representative point setting unit 50 detects setting positions of a plurality of feature points in an ultrasonic image 28. Based on a result of the detection, a recommended area for a position of a feature point which should be set next by the user such as a doctor or a medical technician in manual setting is marked. For example, a recommended area for a position to which the next feature point should be set in the ultrasonic image 28 is marked. For example, as in the specific example shown in FIG. 11, when a position of the first feature point 52a is set, a recommended area for a position corresponding to the second feature point which should be set next in the ultrasonic image 28 is marked in a form of a broken line circle. It goes without saying that the recommended area may also be marked in a form other than a circle.

Then, the user such as a doctor or a medical technician moves an arrow-shaped cursor AC shown in the display image 84 to, for example, a desired position in the recommended area by, for example, operating the operation receiving unit 90 to designate a position of a feature point which should be set next.

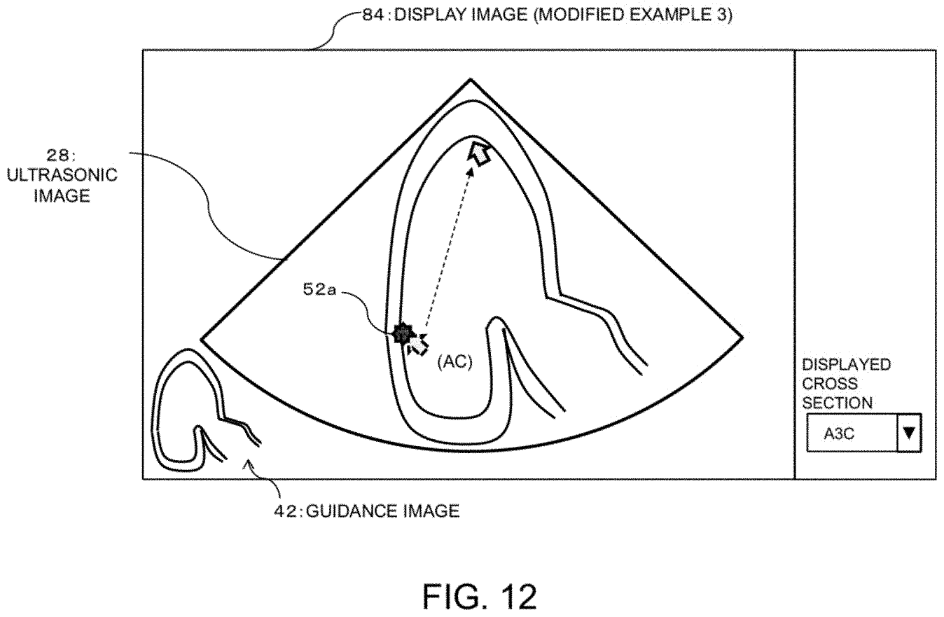

In a modified example 3 shown in FIG. 12, the representative point setting unit 50 detects setting positions of a plurality of feature points in an ultrasonic image 28. Based on a result of the detection, for example, the display image forming unit 80 moves a cursor for setting to a predicted position of a feature point which should be set next by the user such as a doctor or a medical technician in manual setting. For example, as in the specific example shown in FIG. 12, when a position of the first feature point 52a is set, an arrow-shaped cursor AC is moved to a predicted position to which the second feature point which should be set next is to be set. The user such as a doctor or a medical technician slightly adjusts a position of the arrow-shaped cursor AC as necessary by, for example, operating the operation receiving unit 90 to designate a position of a feature point which should be set next.

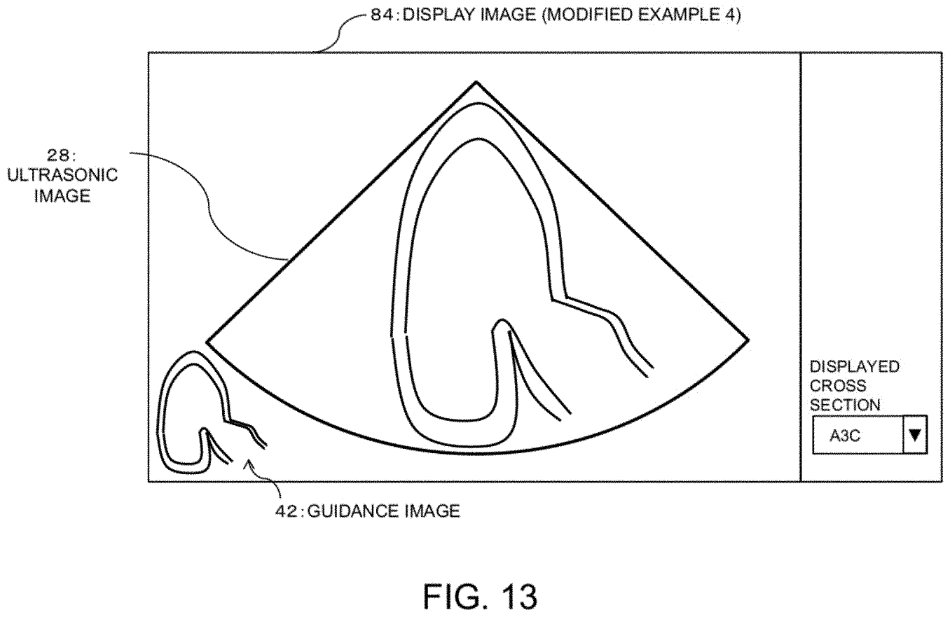

In a modified example 4 shown in FIG. 13, the image type determination unit 30 automatically determines a type of organ image, and a guidance image 42 corresponding to the type of organ image automatically determined by the image type determination unit 30 is displayed. For example, in the case of the diagnosis of the heart, the image type determination unit 30 determines a type corresponding to an ultrasonic image 28 among representative types of tomographic images such as an apical three-chamber (A3C) view, an apical two-chamber (A2C) view, and an apical four-chamber (A4C) view. Then, the guidance image generation unit 40 selects a schema diagram corresponding to the type of organ image determined by the image type determination unit 30 to generate a guidance image 42.

The user may also be able to modify (change) the type of organ image automatically determined by the image type determination unit 30. For example, in the modified example 4 shown in FIG. 13, the user such as a doctor or a medical technician may also operate the menu display for selecting a displayed cross section to select a cross section corresponding to the tomographic image of the heart in the ultrasonic image 28 from the list of displayed cross sections shown in the pull-down menu (see FIG. 2), thereby making it possible to change a type of cross section.

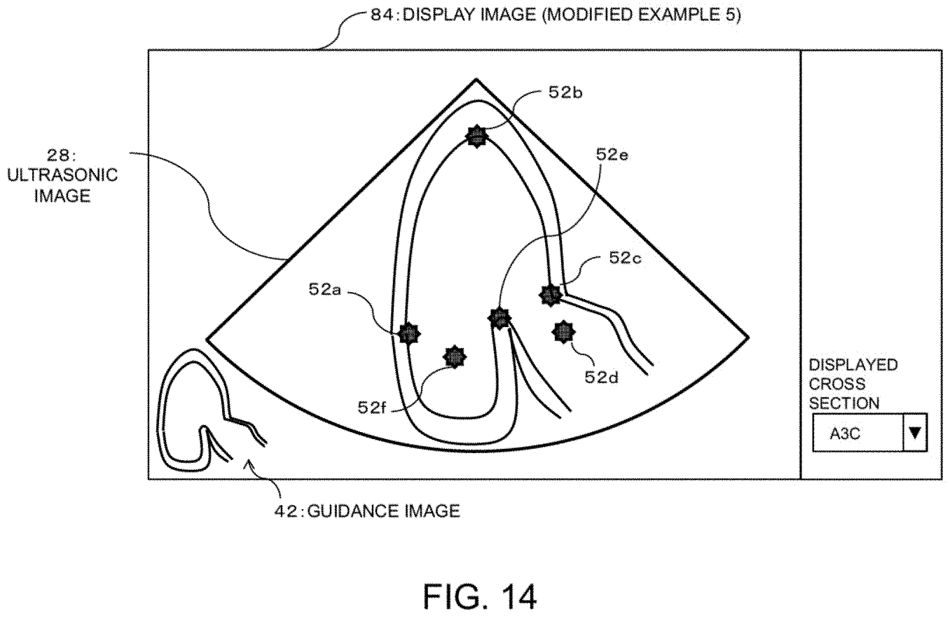

In a modified example 5 shown in FIG. 14, the representative point setting unit 50 detects setting positions of a plurality of feature points in an ultrasonic image 28. Then, a result of the detection of the plurality of feature points by the representative point setting unit 50 is shown in the ultrasonic image 28. For example, as in the specific example shown in FIG. 14, positions corresponding to the plurality of feature points 52a to 52f detected by the representative point setting unit 50 are marked on the ultrasonic image 28.

The user may also be able to modify (change) the positions of the feature points detected by the representative point setting unit 50. For example, in the modified example 5 shown in FIG. 14, the user may also be able to modify positions of the respective feature points 52 marked on the ultrasonic image 28 by operating the operation receiving unit 90 to designate modified positions of the feature points.

Although the preferred embodiments of the present disclosure have been described above, the embodiments described above are merely illustrative in all respects, and do not limit the scope of the present disclosure. The present disclosure includes various modifications without departing from the gist of the present disclosure.

1. An ultrasonic image processing apparatus comprising:
a representative point setting unit which manually sets at least one of a plurality of representative points in an ultrasonic image based on data obtained by transmitting and receiving ultrasonic waves according to an operation by a user; and
an image generation unit which generates a guidance image in which setting position information and setting order information of each manually set representative point are marked on a schematic diagram schematically representing an organ image included in the ultrasonic image.

2. The ultrasonic image processing apparatus according to claim 1, wherein
the image generation unit generates the guidance image in which a position marker as the setting position information and a number label as the setting order information are marked on the schematic diagram.

3. The ultrasonic image processing apparatus according to claim 1, wherein
the image generation unit generates the guidance image corresponding to a type of organ image included in the ultrasonic image by marking setting position information and setting order information corresponding to the type of organ image on the schematic diagram selected according to the type of organ image.

4. The ultrasonic image processing apparatus according to claim 2, wherein
the image generation unit generates the guidance image corresponding to a type of organ image included in the ultrasonic image by marking setting position information and setting order information corresponding to the type of organ image on the schematic diagram selected according to the type of organ image.

5. The ultrasonic image processing apparatus according to claim 1, wherein
the representative point setting unit manually sets one or more representative points inside a bloodstream as representative points for defining an edge of a closed region according to an operation by the user, and
the image generation unit generates the guidance image in which setting position information and setting order information of each representative point manually set inside the bloodstream are marked on the schematic diagram.

6. The ultrasonic image processing apparatus according to claim 5, further comprising:
a tracking point setting unit which sets a plurality of tracking points on the edge of the closed region based on the plurality of representative points; and
a tracking processing unit which tracks movements of the plurality of tracking points over a plurality of time phases by applying a pattern matching processing between time phases based on image data of the ultrasonic image for each tracking point.

7. The ultrasonic image processing apparatus according to claim 6, further comprising:
a vector operation unit which obtains vector information corresponding to one or more positions within the closed region based on movement information of each tracking point obtained by tracking the movements of the plurality of tracking points set on the edge of the closed region, and Doppler information obtained from a plurality of ultrasonic beams passing through the closed region.

8. A program causing a computer to execute functions of:
manually setting at least one of a plurality of representative points in an ultrasonic image based on data obtained by transmitting and receiving ultrasonic waves according to an operation by a user; and
generating a guidance image in which setting position information and setting order information of each manually set representative point are marked on a schematic diagram schematically representing an organ image included in the ultrasonic image.
